(19) United States
(12) Patent Application Publication
(10) Pub. No.: US 2022/0152234 A1
(43) Pub. Date: May 19, 2022

(54) Garment Sanitization Device

(71) Applicant: Bofill Holdings LLC, Opa Locka, FL (US)

(72) Inventor: Peter Bofill, Opa Locka, FL (US)

(21) Appl. No.: 16/953,273

(22) Filed: Nov. 19, 2020

(51) Int. Cl. A61L 2/10; A61L 2/24; A61L 2/26

(52) U.S. Cl. CPC A61L 2/10; A61L 2/24; A61L 2/26; A61L 2202/26; A61L 2202/122; A61L 2202/121; A61L 2202/14; A61L 2202/11

(57) ABSTRACT

A garment sanitization device having a garment chamber and a sanitization unit. The garment chamber includes a first appendage, a second appendage, and a fastening mechanism. The sanitization unit includes a light assembly and an actuation mechanism. The light assembly is configured to emit sterilizing wavelengths of light, such as ultraviolet light, and is operably coupled to the actuation mechanism, such that when the actuation mechanism is triggered, the light assembly is switched from an off state to an on state. The light assembly is oriented to emit light within the garment chamber. The sanitization unit may further include a timer for regulating the amount of light exposure and a processing unit for operably controlling the light assembly. The garment chamber may further include a view port that allows the user to ensure the light assembly is in the on state.

### B. CROSS-REFERENCES TO RELATED APPLICATIONS

None.

### C. STATEMENT REGARDING FEDERAL SPONSORED RESEARCH OR DEVELOPMENT

None.

### D. NAMES OF PARTIES TO A JOINT RESEARCH AGREEMENT

None.

### E. REFERENCE TO A “SEQUENCE LISTING”, A TABLE, OR A COMPUTER PROGRAM LISTING APPENDIX SUBMITTED ON COMPACT DISC AND INCORPORATION-BY-REFERENCE OF THE MATERIAL ON THE COMPACT DISCLOSURE

None.

### F. STATEMENT REGARDING PRIOR DISCLOSURES BY AN INVENTOR OR JOINT INVENTOR

None.

### G. BACKGROUND OF THE INVENTION

### 1. Field of the Invention

The present invention relates to garments, and more particularly, to an improved device and method for sanitizing garments.

### 2. Description of Related Art Including Information Disclosed Under 37 CFR 1.97 and 37 CFR 1.98

Several designs for garment transportation devices have been designed in the past. None of them, however, includes means for sanitizing the garments. Traditionally, luggage is utilized to transport garments when an individual is traveling. Most traditional luggage requires an individual to fold their clothing in some way, which can lead to wrinkles or otherwise disfiguration of the clothing when unpacking. Thus, additional luggage types, such as garment bags, have been designed for higher-end garments, such as suits, dresses, tuxedos, coats, etc. Such higher-end garment bags allow the garments to rest in a hanging position and thus avoid the any wrinkling or other disfiguration. While both traditional luggage and higher-end garment bags provide a suitable means for transporting garments from one location to another, they do not provide any means for sanitizing said garments.

Other patents describing the closest subject matter provide for a number of more or less complicated features that fail to solve the problem in an efficient and economical way. None of these patents suggest the novel features of the present invention.

A brief abstract of the technical disclosure in the specification and title are provided as well for the purposes of complying with 37 CFR 1.72 and are not intended to be used for interpreting or limiting the scope of the claims.

Without limiting the scope of the invention, a brief summary of some of the claimed embodiments of the invention is set forth below. Additional details of the summarized embodiments of the invention and/or additional embodiments of the invention may be found in the detailed description of the invention below.

### H. BRIEF SUMMARY OF THE INVENTION

It is one of the main objects of the present invention to provide a device for sanitizing clothing.

It is another object of this invention to provide a garment sanitization device which is free of solvents, abrasives, or other means that could damage the material of the clothing.

It is still another object of the present invention to provide a means for sanitizing clothing using light irradiation and that is energy efficient.

It is yet another object of this invention to provide such a garment sanitization device that is inexpensive to manufacture and maintain while retaining its effectiveness.

Further objects of the invention will be brought out in the following part of the specification, wherein detailed description is for the purpose of fully disclosing the invention without placing limitations thereon.

These and other embodiments which characterize the invention are pointed out with particularity in the claims annexed hereto and forming a part hereof. However, for a better understanding of the invention, its advantages and objectives obtained by its use, reference can be made to the drawings which form a further part hereof and the accompanying descriptive matter, in which there are illustrated and described various embodiments of the invention.

### I. BRIEF DESCRIPTION OF THE SEVERAL VIEWS OF THE DRAWING

With the above and other related objects in view, the invention exists in the details of construction and combination of parts as will be more fully understood from the following description, when read in conjunction with the accompanying drawings in which:

FIG. 1 shows a front plan view of a garment sanitization device, wherein a light assembly is configured to illuminate the interior of a garment chamber.

FIG. 2 shows a front sectional view of the garment chamber, wherein the light assembly and a reflective surface is integrated across the garment chamber.

FIG. 3 shows a front plan view of a garment sanitization device, wherein a charging device is connected to the garment chamber in order to power the sanitization unit.

### J. DETAILED DESCRIPTION OF THE INVENTION

While this invention may be embodied in many different forms, there are described in detail herein specific embodiments of the invention. This description is exemplary of the principles of the invention and is not intended to limit the invention to the particular embodiments illustrated and described.

For the purpose of this disclosure, like reference numerals in the figures shall refer to like features unless otherwise indicated or is obvious by context.

The subject garment sanitization device is sometimes referred to as the device, the invention, machine or other similar terms. These terms may be used interchangeably as context requires and from use the intent becomes apparent. The masculine can sometimes refer to the feminine and neuter and vice versa. The plural may include the singular and singular the plural as appropriate from a fair and reasonable interpretation in the situation.

Referring now to the drawings, where the present invention is generally referred to with numeral 10, it can be observed that it basically includes a garment chamber 11 and a sanitization unit 20. The garment chamber 11 is intended to receive one or more pieces of clothing at a time and generally comprises a first appendage 12, a second appendage 13, and a fastening mechanism 14. Meanwhile, the sanitization unit 20 is intended to sterilize the clothing positioned within the garment chamber 11. The sanitization unit 20 generally comprises a light assembly 21 and an actuation mechanism 22. Preferably the present invention is configured to be a portable device that can be used to transport clothing; however, the present invention may also be configured as a more permanent fixture for use in spaces such as retail stores or household bedrooms.

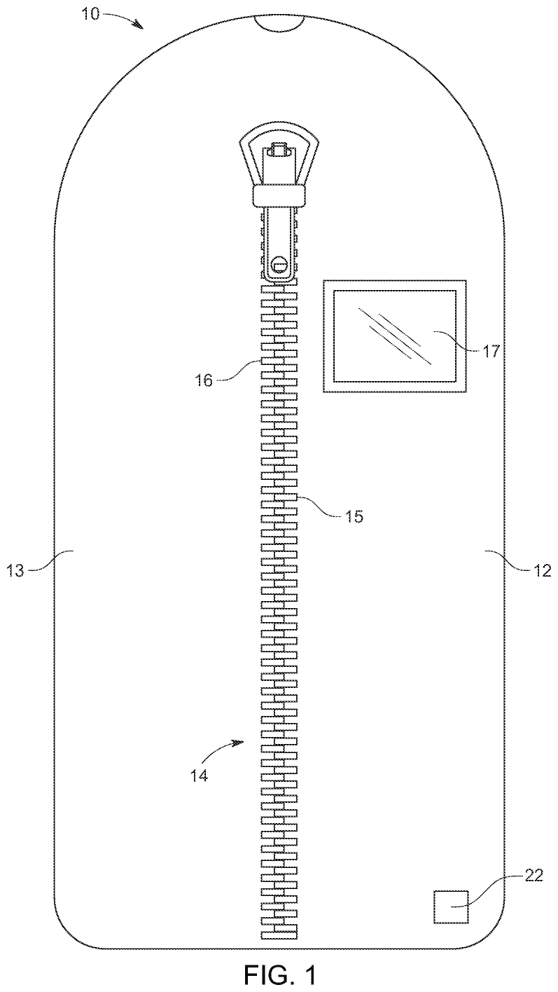

In reference to FIG. 1, the garment chamber 11 is an enclosure, wherein the first appendage 12 and the second appendage 13 form a sealable opening for placing clothing into or removing clothing from the garment chamber 11. As such, the first appendage 12 is positioned adjacent to the second appendage 13. In the preferred embodiment, the garment chamber 11 is a soft-body enclosure, such that the garment chamber 11 can be rolled, folded, or otherwise contorted to a smaller formfactor for transport, storage, etc. Furthermore, in the preferred embodiment, the first appendage 12 and the second appendage 13 are flap-like members that can be contorted away from each other to create an opening for accessing the interior of the garment chamber 11. In other embodiments, the garment chamber 11 may be a hard-body enclosure or a hybrid soft-body and hard-body enclosure. Thus, in some embodiments, the first appendage 12 and/or the second appendage 13 may be a hinged member more similar to a door.

The fastening mechanism 14 is integrated with the first appendage 12 and the second appendage 13, wherein the fastening mechanism 14 provides a means for selectively sealing the garment chamber 11 closed. The fastening mechanism 14 comprises a first fastener 15 and a second fastener 16, wherein the first fastener 15 is positioned along the first appendage 12 and the second fastener 16 is positioned along the second appendage 13. Furthermore, the first fastener 15 is positioned adjacent to the second fastener 16, wherein the first fastener 15 is selectively engaged with the second fastener 16 in order to open and close the garment chamber 11. In the preferred embodiment, the fastening mechanism 14 is a zipper fastener, wherein the first fastener 15 and the second fastener 16 are each a row of offset teeth. In other embodiments, the fastening mechanism 14 may be a hook and loop fastener, buttons, snaps, etc., wherein the first fastener 15 and the second fastener 16 are opposable mating members that are releasably engaged with each other.

In reference to FIG. 1, in the preferred embodiment, the first appendage 12 and the second appendage 13 are coplanar, wherein the first appendage 12 and the second appendage 13 form the front half of the garment chamber 11. As such, the fastening mechanism 14 is accessible along the frontside of the garment chamber 11. In other embodiments, the first appendage 12 and the second appendage 13 may form separate halves of the garment chamber 11. For example, the first appendage 12 may form the frontside of the garment chamber 11 while the second appendage 13 forms the backside of the garment chamber 11. In such embodiments, the first appendage 12 and the second appendage 13 may be permanently affixed to each other along one or more opposing edges. Meanwhile, the fastening mechanism 14 is positioned along one or more non-affixed edges of the first appendage 12 and the second appendage 13. In this way, the fastening mechanism 14 is positioned along one ore more of the sides, top, or bottom of the garment chamber 11.

The garment chamber 11 further comprises a superior opening that is positioned above the fastening mechanism 14. The superior opening allows the hook portion of a hanger to pass through the garment chamber 11, wherein the hanger supports both the clothing and the garment chamber 11. The superior opening may be reinforced along the edges in order to prevent the garment chamber 11 from tearing about the superior opening.

The garment chamber 11 may further comprise one or more handles. The one or more handles provides the user with a means for grasping the garment chamber 11 in order to transport the garment chamber 11. Furthermore, the one or more handles can be used as a support to suspend the garment chamber 11 from a hook, doorknob, etc. In one embodiment, a first handle is positioned about the top of the garment chamber 11 while a second handle is positioned about the bottom of the garment chamber 11. In this way the garment chamber 11 can be folded over and carried in a manner similar to that of a suitcase.

The garment chamber 11 may further comprise on or more retaining members. The one or more retaining members allows the garment chamber 11 to be secured in a compact configuration, such as being folded over or rolled up. The one or more retaining members may be hook and loop fasteners, snap locks, buckles, magnets, or any other similar mechanisms. In one embodiment, a first retaining member and a second retaining member are positioned about the top of the garment chamber 11, while a third retaining member and a fourth retaining member are positioned about the bottom of the garment chamber 11. When the garment chamber is folded over, the first retaining member is engaged with the third retaining member and the second retaining member is engaged with the fourth retaining member, wherein the garment chamber 11 is secured in the folded position. The corresponding retaining members are then disengaged to unfold the garment chamber 11.

The sanitization unit 20 is integrated with the garment chamber 11, wherein the light assembly 21 is configured to illuminate the interior of the garment chamber 11 in order to irradiate the clothing. The light assembly 21 may be integrated with one or more of the first appendage 12, the second appendage 13, or any other interior surfaces of the garment chamber 11. The light assembly 21 is configured to emit sterilizing wavelengths of light, such as ultraviolet light. In the preferred embodiment, the light assembly 21 is configured to emit ultraviolet C light which is the best-known wavelength for effectively performing germicidal irradiation. However, different wavelengths may be used in other embodiments, as the different wavelengths may be found to be more effective depending on the material of the garment or the environment in which the device is placed.

In general, the light assembly 21 comprises a plurality of lighting elements, wherein the plurality of lighting elements is distributed about the interior of the garment chamber 11. Preferably each of the plurality of lighting elements is a small form factor device, such as a light emitting diode (LED) light strip. This allows the garment chamber to maintain a slimmer profile and makes the garment chamber easier to transport. However, it is possible for each of the plurality of lighting elements to be any other suitable type of lighting element capable of emitting irradiating light, such as an incandescent bulb, fluorescent bulb, or halogen bulb. The specific number of the plurality of lighting elements used depends on the type of light elements, the intensity of the light elements, and the placement of the light elements about the garment chamber 11.

The light assembly 21 is electrically connected to the actuation mechanism 22, wherein the actuation mechanism 22 toggles the light assembly 21 between an on state and an off state. In the on state, the light assembly 21 is emits ultraviolet C light, or another irradiating wavelength of light. The actuation mechanism 22 is integrated with the garment chamber 11 such that the actuation mechanism 22 may be engaged by a user or such that the actuation mechanism 22 is able to detect the presence of clothing within the garment chamber 11. When the actuation mechanism 22 is engaged or detects the presence of clothing, the light assembly 21 is toggled to the on state. Subsequently, when the actuation mechanism 22 is engaged a second time or the presence of the clothing is no longer detected, the light assembly 21 is toggled to the off state.

In some embodiments, the actuation mechanism 22 is a push button, flip switch, or similar mechanism, wherein the actuation mechanism 22 is engaged a first time to switch the light assembly 21 to the on state and engaged a second time to switch the light assembly 21 to the off state. Preferably the actuation mechanism 22 is integrated with the exterior of the garment chamber 11 such that a user may engage the actuation mechanism 22 with their hand or finger.

In other embodiments, the actuation mechanism 22 may be integrated with the fastening mechanism 14. The actuation mechanism 22 is integrated with the fastening mechanism 14 such that when the fastening mechanism 14 is fully closed to seal the garment chamber 11 shut, the light assembly 21 is switched from the off state to the on state. Conversely, when the fastening mechanism 14 is opened, the light assembly 21 is switched from the on state to the off state. For example, if the fastening mechanism 14 is a zipper, then the actuation mechanism may be a flip switch that is engaged by the zipper pull. As another example, if the fastening mechanism 14 is a hook and loop fastener, then the actuation mechanism may be an electromagnetic switch, near field communication (NFC) device, radio frequency identification (RFID) device, etc.; the actuation mechanism comprises a first actuator and a second actuator, wherein one is integrated with the first fastener 15 and the other is integrated with the second fastener 16 such that when the first fastener 15 and the second fastener 16 engage each other the first actuator and the second actuator are positioned adjacent each and communicate with one another.

In some embodiments, the sanitization unit 20 may further comprise a timer 23 to automatically switch the light assembly 21 from the on state to the off state. In this way, the user must only engage the actuation mechanism 22 a single time. Further, this limits the duration for which the light assembly 21 is switched to the on state and thus increases the energy efficiency of the present invention. The timer 23 is electrically connected with the light assembly 21 such that the timer 23 breaks the circuit after a preset time in order to toggle the light assembly 21 from the on state to the off state. In some embodiments the timer 23 may be integrated with the actuation mechanism 22, wherein the timer 23 flips the actuation mechanism 22 in order to break the circuit. In other embodiments, the timer 23 may have its own independent means for breaking the circuit.

The sanitization unit 20 may further comprise a processing unit 24, wherein the processing unit 24 is electronically connected to the actuation mechanism 22 and the light assembly 21. The actuation mechanism 22 and the processing unit 24 are configured to detect the presence of clothing within the garment chamber 11. When the presence of clothing is detected, the processing unit 24 toggles the light assembly 21 to the on state.

In one embodiment, the actuation mechanism 22 may be a trip sensor, such as a laser tripwire. The light assembly 21 is normally in the off state and when the actuation mechanism 22 is tripped, the processing unit 24 switches the light assembly 21 to the on state. In such embodiments, the actuation mechanism 22 is integrated with the interior of the garment chamber 11, wherein the actuation mechanism 22 is comprises the first actuator and the second actuator. The first actuator and the second actuator are positioned opposite each other within the garment chamber 11, such that when clothing is positioned within the garment chamber 11, the clothing is positioned in between the first actuator and the second actuator. For example, the first actuator may be integrated with the frontside of the garment chamber 11 while the second actuator is integrated with the backside of the garment chamber. When the clothing is placed between the first actuator and the second actuator, the communication between the first actuator and the second actuator is broken, wherein the light assembly 21 is triggered and switched to the on state by the processing unit 24. When the clothing is removed from the garment chamber 11, the communication between the first actuator and the second actuator is re-established and the light assembly 21 is switched to the off state by the processing unit 24.

In another embodiment, the actuation mechanism 22 is a transceiver. The actuation mechanism 22 allows the processing unit 24 to communicate with an external device, such as a smartphone, NFC tag, RFID tag, etc. When the processing unit 24 detects the presence of the external device via the actuation mechanism 22, the processing unit 24 switches the light assembly 21 to the on state.

When the actuation mechanism 22 is a transceiver, the external device can also be used to remotely adjust settings of the sanitization unit 20. A control application may be installed on the external device, wherein a user is able to manipulate or monitor settings for the light assembly 21 remotely through the control application. For example, the control application may allow the user to adjust the intensity of the light assembly 21, toggle the light assembly 21 between the on state and the off state, or monitor the electricity consumption of the light assembly 21.

In some embodiments the sanitization unit 20 may further comprise a battery. The battery lends to the portability of the present invention and allows the present invention to be used in almost any setting. The battery is electrically connected to the light assembly 21, the actuation mechanism 22, the timer 23, and/or the processing unit 24. The battery may be replaceable or rechargeable depending on the embodiment. When the battery is rechargeable, a charging port may be provided in the garment chamber 11, such that a charging cable can be removably attached in between the battery and an outlet or other power source. In this way, the battery does not need to be removed from the garment chamber 11 in order to be charged.

In other embodiments, the sanitization unit 20 may comprise a power converter and a charging cable instead of a battery. The charging cable allows the sanitization unit 20 to be connected to an outlet or other power source while the power converter regulates the current and supplies power to the light assembly 21, the actuation mechanism 22, the timer 23, and/or the processing unit 24. The charging cable may be removably attached to the power converter or permanently connected to the power converter. Particularly in embodiments when the charging cable is permanently connected to the power converter, the power cable may be retractable, such that the power cable can be stored within the garment chamber 11 when not in use.

In some embodiments, the processing unit 24 may be used to control an illumination pattern of the light assembly 21. The illumination pattern may include turning the plurality of lighting elements on row-by-row, column-by-column, frontside-to-backside, top-to-bottom, etc. This may allow for a lower power load on the battery and thus allow for the battery to have a smaller form factor, which would in turn increase the portability of the present invention.

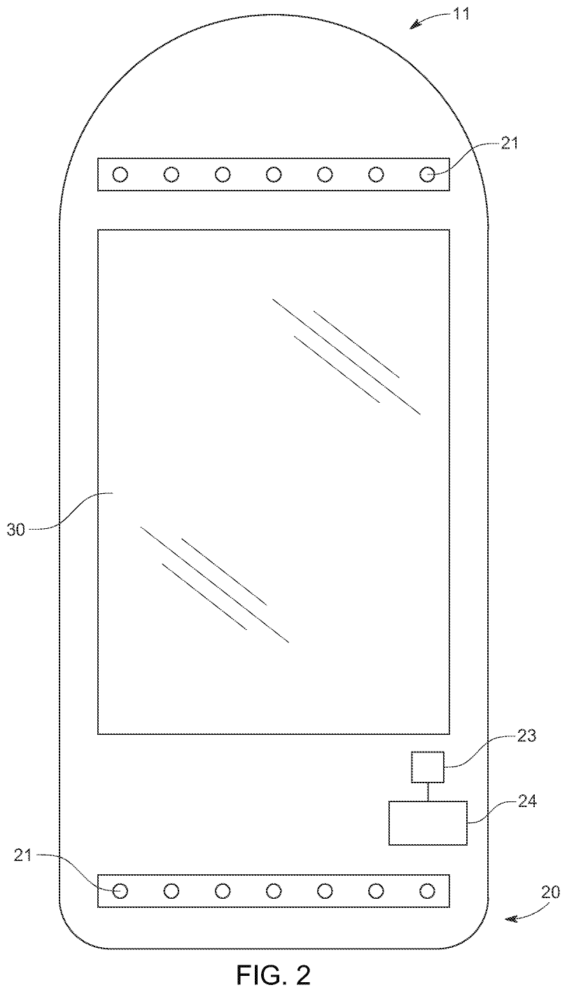

In reference to FIG. 2, the present invention may further comprise a reflective surface 30. The reflective surface 30 is integrated with one or more interior surfaces of the garment chamber 11 such that the reflective surface 30 reflects the light emitted by the light assembly 21. In this way, the number of lighting elements of the light assembly 21 may be reduced, which in effect reduces energy consumption and may reduce manufacturing costs. The reflective surface 30 may also allow for a lower power load on the battery and thus allow for the battery to have a smaller formfactor, which would in turn increase the portability of the present invention.

In some embodiments, the light assembly 21 may comprise one or more operable mounts, wherein each of the operable mounts supports one or more lighting elements. The operable mounts may allow for simple movement of the lighting elements, such as a pivoting motion, or more advanced motion, such as moving along a track, wherein the lighting elements can be relocated within the garment chamber 11. Each of the operable mounts is electronically connected to the processing unit 24 such that the processing unit 24 may adjust the direction of the light emitted from the lighting element. In this way, the light assembly 21 can be targeted to specific regions of the garment chamber 11. This also allows the light assembly 21 to be used in a scanning manner, wherein the number of lighting elements can be reduced. For example, a single lighting element can be mounted to a track that runs across the top of the garment chamber 11; the single lighting element can then be moved side to side in order to irradiate the clothing.

The sanitization unit may further comprise a charging device 25 that is electrically connected to the light assembly 21. In some embodiments, the charging device 25 may be directly connected to the light assembly 21. In this way, the charging device 25 can be used to power the light assembly 21 without the use of the battery or power converter. In other embodiments, the charging device 25 may be connected to the light assembly 21 via the battery, wherein the charging device 25 is used to recharge the battery. The charging device 25 may also be connected to the light assembly 21 or the battery via the charging cable.

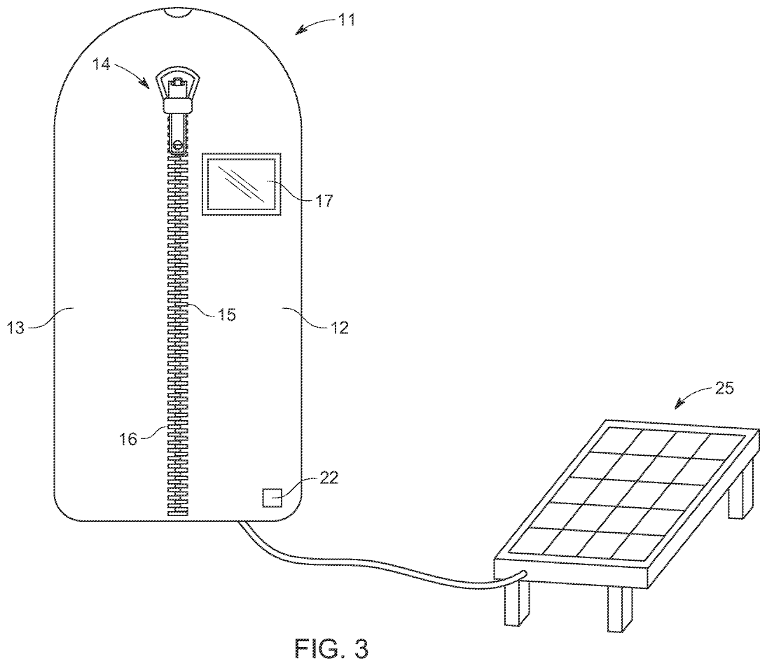

In reference to FIG. 3, in some embodiments, the charging device 25 is a solar panel. The solar panel is preferably integrated with the exterior of the garment chamber 11. In some embodiments, the solar panel may be permanently integrated with the garment chamber 11, wherein the present invention can be placed in a sunlit location, such as a window, in order to generate power via the solar panel. As such, the garment chamber 11 may further include a hanger, wherein the hanger allows the garment chamber 11 to be suspended by another supporting object. Exemplary embodiments of the hanger include, but are not limited to, a traditional hook, a loop, an adhesive, or one or more suction cups.

In other embodiments, the solar panel may be removably attached to the garment chamber 11, such that the solar panel can be placed in a sunlit location independent of the garment chamber 11. The solar array may be held in place about the exterior of the garment chamber 11 by a snap lock, press fit, magnetic connectors, or any other suitable means for temporarily securing the solar panel to the garment chamber 11. In such embodiments, the solar panel may be connected to the light assembly 21 or the battery via the charging cable, such that the solar panel can be extended away from the garment chamber 11 and generate power for the light assembly 21. To better position the solar panel in the sunlit location, the solar panel may comprise a panel hanger such as a hook, a loop, an adhesive, or one or more suction cups. The solar panel may also comprise a panel support that can be used to adjust the height or angle of the solar panel.

The panel support may be configured between a stowed position and an un-stowed position. In the stowed position, the panel support is retracted, collapsed, detached, or otherwise configured to reduce the profile of the solar panel. In some embodiments, the panel support may be a telescoping leg, a hinged leg, or otherwise retractable or collapsible member, wherein the panel support is able to be retracted into a main body of the solar panel. In other embodiments, the panel support may be detached from the main body and stowed in a storage area integrated in either the garment chamber 11 or the main body. In such embodiments, the panel support may be attached to the main body by a threaded connection, ball lock pin, press fit, snap lock, or any other suitable means of connection.

In other embodiments, the charging device 25 is a piezoelectric device. The piezoelectric device may be positioned about the interior or exterior of the garment chamber 11 or piezoelectric device may be integrated directly with the garment chamber 11. In one embodiment, the piezoelectric device is a plurality of piezoelectric transducers integrated throughout the garment chamber 11, wherein the garment chamber 11 is constructed from a flexible material. In this way, as the garment chamber is jostled around, either purposefully or during transport, the piezoelectric device generates power. In another embodiment, the piezoelectric device is a hand-held apparatus that is electrically connected to the light assembly 21 or the battery via the charging cable. A user may hold and shake, or otherwise articulate, the piezoelectric device in order to generate electricity. When not in use, the piezoelectric device may be stored on or within the garment chamber 11.

In reference to FIG. 1, in some embodiments, the garment chamber 11 may further comprise a view port 17. The view port 17 may be integrated with the first appendage 12, the second appendage 13, or any other exterior of the garment chamber 11. The view port 17 allows a user to observe the status of the light assembly 21. More specifically, the view port 17 allows a user to observe if the light assembly 21 is switched to the on state or the off state. This allows the user to have peace of mind that the clothing is being sanitized or that the battery is not being inadvertently depleted.

In some embodiments, the view port 17 is formed by a grommet. In such an embodiment, the grommet reinforces the opening formed in the garment chamber 11 and provides a rigid or semi-rigid structure that maintains the hole in the garment chamber 11 in an open position, such that a user can view inside the garment chamber 11. The view port 17 may also comprise a transparent sheet, wherein the grommet supports the transparent sheet about the hole in the garment chamber 11. In this way, the garment chamber 11 remains fully enclosed but the user is still able to view the interior of the garment chamber 11. The transparent sheet may be fully transparent or semi-transparent, so long as enough light is able to pass through and allow the user to differentiate between the on state and the off state.

Furthermore, the transparent sheet may be rigid or flexible. The transparent sheet being rigid may lend to easier viewing of the interior of the garment chamber 11, as the transparent sheet would maintain the hole in the garment chamber 11 in an open position. On the other hand, the transparent sheet being flexible may lend to the garment chamber 11 being more compactable, as it would be easier to fold, roll, or otherwise stow the view port 17 portion along with the body of the garment chamber 11.

As an alternative to the view port 17, in some embodiments the garment chamber 11 may be constructed from a transparent or semi-transparent material. This would allow the user to view the interior of the garment chamber 11 from all angles and ensure the light assembly 21 is fully operational. A semi-transparent material would be preferable over a fully transparent material, as less light would be permitted to escape, thus lending to a higher energy efficiency and exposing the user to less radiance.

The foregoing description conveys the best understanding of the objectives and advantages of the present invention. Different embodiments may be made of the inventive concept of this invention. It is to be understood that all matter disclosed herein is to be interpreted merely as illustrative, and not in a limiting sense.

I/We claim:

1. A garment sanitization device, the garment sanitization device comprising:
a garment chamber; the garment chamber comprising a first appendage, a second appendage, and a fastening mechanism; the fastening mechanism being integrated with the first appendage and the second appendage; the fastening mechanism comprising a first fastener being positioned along the first appendage and a second fastener being positioned along the second appendage; the first fastener being positioned adjacent to the second fastener, wherein the first fastener is selectively engaged with the second fastener;
a sanitization unit being integrated with the garment chamber; the sanitization unit comprising a light assembly and an actuation mechanism; the light assembly being electrically connected to the actuation mechanism, wherein the light assembly can be toggled between an on state and an off state; the light assembly being configured to illuminate the interior of the garment chamber; and the light assembly being configured to emit ultraviolet light in the on state.

2. The garment sanitization device as claimed in claim 1 further comprising:
a timer;
the timer being electrically connected with the light assembly; and the timer being configured to toggle the light assembly from the on state to the off state after a preset time.

3. The garment sanitization device as claimed in claim 1 further comprising:
a processing unit;
the processing unit being electronically connected to the actuation mechanism and the light assembly; and
the processing unit toggling the light assembly to the on state when the actuation mechanism is triggered.

4. The garment sanitization device claimed in claim 3, wherein the actuation mechanism is integrated with the fastening mechanism.

5. The garment sanitization device as claimed in claim 1 further comprising:
a charging device;
the charging device being electrically connected to the light assembly.

6. The garment sanitization device as claimed in claim 7, wherein the charging device is a solar panel.

7. The garment sanitization device as claimed in claim 7, wherein the charging device is a piezoelectric device.

8. The garment sanitization device as claimed in claim 1 further comprising:
the garment chamber further comprising a view port; the view port being integrated with the first appendage, wherein ultraviolet light is visible through the view port when the light assembly is in the on state.

9. The garment sanitization device as claimed in claim 1 further comprising:
a reflective surface; and the reflective surface being interiorly positioned about the garment chamber.
